(19) United States
(12) Patent Application Publication
(10) Pub. No.: US 2016/0310755 A1
(43) Pub. Date: Oct. 27, 2016

(54) Sunbed Tanning

(71) Applicant: Innovate Photonics Limited, Swansea (GB)

(72) Inventor: David Simon George, Swansea (GB)

(21) Appl. No.: 14/718,289

(22) Filed: May 21, 2015

(30) Foreign Application Priority Data
Apr. 22, 2015 (GB) 1506834.9

(51) Int. Cl. A61N 5/06

(52) U.S. Cl. CPC A61N 5/0614; A61N 2005/0615; A61N 2005/0651; A61N 2005/0663; A61N 2005/0661

(57) ABSTRACT

A sunbed with at least one flexible substrate onto or between which a person requiring a sun tan may res. The or each substrate may have therein or thereon an array of LEDs covering substantially the whole of at least one side of the substrate whereby, in use, the LEDs are able to provide an even coverage of UV light to a user of the sunbed.

This invention relates to sunbeds of the type that provide a source of ultra-violet (UV) radiation for the purposes of tanning skin and photo-rejuvenation.

A typical sunbed comprises an elongate array of tubes for producing light in the ultra-violet part of the electromagnetic spectrum, each tube typically being rated between 160 to 250 watts in terms of electrical power consumption but having an efficiency of perhaps just 2-3%. Accordingly, an array of such tubes arranged to tan the front, back and sides of the human form can typically consume about 10 kilowatts of power and, in doing so, the tubes emit UVA rays in the 320-400 nm range and UVB rays in the 290-320 nm range, this latter range being known for causing skin to burn and therefore being responsible for the development of melanoma or skin cancer. Such traditional sunbeds, requiring multiple UV tubes with a closable bed structure designed to protect them from direct contact with a user of the bed whilst the user is supported thereon or therein, are expensive to manufacture and run, and are generally indiscriminate as to which type of UV light is exposed to the skin of the user of the bed.

The present invention is derived from the realisation that a different approach to artificial tanning or photo-rejuvenation is required which is cheaper to manufacture than traditional tube-type sunbeds as well as being more efficient and less costly to run.

According to the invention there is provided a sunbed comprising at least one flexible substrate onto or between which a person requiring a sun tan may rest, the or each substrate having therein or thereon an array of LEDs covering substantially the whole of at least one side of the substrate whereby, in use, the LEDs are able to provide an even coverage of UV light to a user of the sunbed.

With this arrangement, a simple mechanical structure can be used to support the sunbed during use which may thereafter be rolled up for storage, as compared to conventional rigid sunbeds which use up a large space even when not in use. Alternatively, the sunbed may be in the form of a blanket or rug supported by the user or on any convenient surface.

Preferably, the or each substrate only uses LEDs that provide UVA radiation, such that there is no or little risk of the skin burning. Whilst LEDs operating in the 365 nm region, which emit UVA, are known to be less efficiently absorbed than LEDs operating in the 335 nm UVB region of the spectrum, nevertheless it will be appreciated that because there are a large number of them, they are still capable of e.g. tanning or rejuvenating skin, but in a safer way which does not involve the danger of the skin burning.

Conveniently, the or each substrate also includes an array of LEDs operable in the visible 635 nm (red) wavelength or any other suitable wavelength.

The invention will now be described, by way of example only, with reference to the accompanying drawings in which:

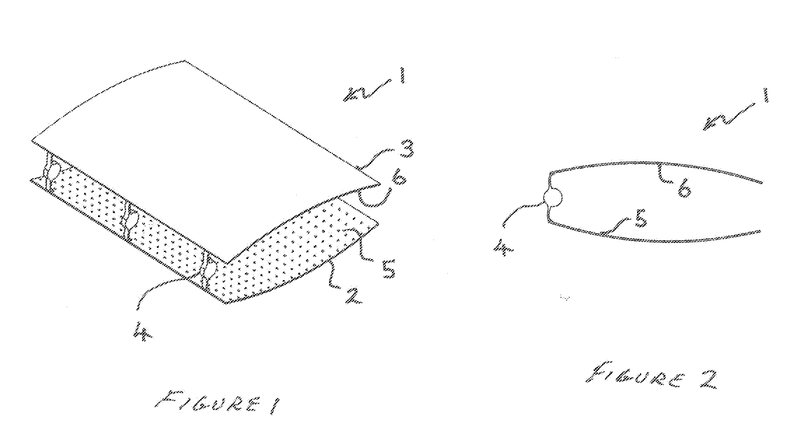

FIG. 1 is a perspective view of a 2-part sunbed according to the invention

FIG. 2 is an end view of the sunbed of FIG. 1.

Referring to the drawings, a sunbed shown generally at 1 comprises a lower half 2 and an upper half 3, each being of e.g. acrylic and being connected at one side via hinges 4.

The upper side 5 of the lower half 2 and the lower side 6 of the upper half 3 are each provided with a flexible substrate having an array of LEDs, typically 1000 per side, the LEDs themselves being of the type delivering UVA or visible 635 nm radiation. The LEDs of each substrate are electrically connected via a flexible circuit board to a power supply and associated switch (not shown).

As will be apparent, because the substrates 5,6, are flexible the lower substrate 5 can be placed on a simple mechanical support structure, whereafter a user of the sunbed can lie or sit on top whereafter the upper substrate 6 can be closed around the user, thereby providing an array of LEDs that closely conform to the shape of the body, essentially being next to the skin as compared to the source of UV radiation being spaced from the skin with traditional sunbeds using UV tubes. This elegantly simple arrangement means that LEDs operating only at the UVA level can be used to thereby avoid the risk of burning of the skin whilst still allowing a uniform tan to be developed or whilst still allowing for photo-rejuvenation of the skin, as the case may be. In the case of the latter there may be an assortment of LEDs on or in each substrate, one kind producing UVA and another kind producing 635 nm (red) light, each type being used in combination with the other, or being selectively used separately, as required. The invention therefore provides a relatively inexpensive solution to the problem of artificially obtaining the benefits of UV radiation using inexpensive LEDs having a typical lifetime of perhaps 20,000 hours as compared to UV tubes which are expensive to manufacture and have a lifetime of only around 500 hours, also requiring substantial bespoke support apparatus and occupying a substantial amount of space. In contrast, a flexible sunbed of the type described does not require a large support structure and can therefore be substantially smaller than existing products.

1. A sunbed comprising at least one flexible substrate onto or between which a person requiring a sun tan may rest, the or each substrate having therein or thereon an array of LEDs covering substantially the whole of at least one side of the substrate whereby, in use, the LEDs are able to provide an even coverage of UV light to a user of the sunbed.

2. A sunbed according to claim 1 wherein the or each substrate only uses LEDs that provide UVA radiation, such that there is no or little risk of the skin burning.

3. A sunbed according to claim 1 wherein the or each substrate also includes an array of LEDs operable in the visible 635 nm (red) wavelength or any other suitable wavelength.

4. A sunbed according to claim 2 wherein the or each substrate also includes an array of LEDs operable in the visible 635 nm (red) wavelength or any other suitable wavelength.
